(19) United States
(12) Patent Application Publication
(10) Pub. No.: US 2010/0029743 A1
(43) Pub. Date: Feb. 4, 2010

(54) ATORVASTATIN PHARMACEUTICAL COMPOSITIONS

(75) Inventors: Sudeep Kumar Agrawal, Raipur (IN); Abhijeet Ashok Upadhye, Aurangabad (IN); Indu Bhushan, Hyderabad (IN); Mailatur Sivaraman Mohan, Hyderabad (IN); Riswan Zafar, Kishanganj (IN); Alagumurugan Alagarsamy, Tuticorin (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500 016, Andhra Pradesh (IN); DR. REDDY'S LABORATORIES, INC., Bridgewater, NJ (US)

(21) Appl. No.: 12/443,242

(22) PCT Filed: Sep. 27, 2007
(86) PCT No.: PCT/US07/79641

§ 371 Date: Mar. 27, 2009

Related U.S. Application Data
(60) Provisional application No. 60/883,093, filed on Jan. 2, 2007.

(30) Foreign Application Priority Data
Sep. 27, 2006 (IN) 1790/CHE/2006

(51) Int. Cl. A61K 31/40; A61P 9/00

(52) U.S. Cl. 514/423

(57) ABSTRACT

The present invention relates to pharmaceutical formulations of atorvastatin or its pharmaceutically acceptable salts, solvates, hydrates, enantiomers, polymorphs or their mixtures; and processes for preparing the same, and their methods of use, treatment and administration. Further, the present invention relates to pharmaceutical compositions comprising an acid-solubility-enhanced form of atorvastatin or its salts.

### INTRODUCTION TO THE INVENTION

The present invention relates to pharmaceutical compositions of atorvastatin or its pharmaceutically acceptable salts, solvates, hydrates, enantiomers, polymorphs or their mixtures; and processes for preparing the same, their methods of use, treatment and administration.

The present invention further relates to pharmaceutical compositions comprising a solubility-enhanced form of atorvastatin, such composition having a specific in-vitro release pattern in acidic and alkaline pH conditions of the gastrointestinal system, thus ensuring desired in-vivo profiles.

Atorvastatin calcium, a potent molecule from the “statin” family, is a lipid-lowering agent that acts by inhibiting the HMG-CoA reductase enzyme. Atorvastatin calcium has a chemical name [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate and has structural Formula I.

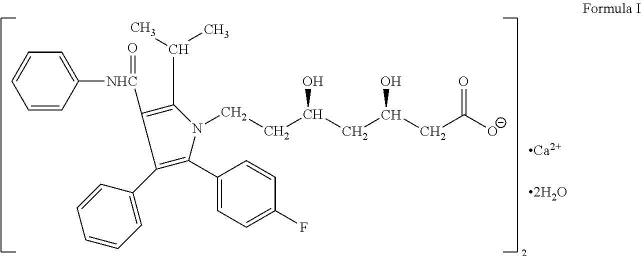

A commercially available product containing atorvastatin is LIPITOR® oral tablets, manufactured by Pfizer. LIPITOR® tablets contain atorvastatin as its calcium salt trihydrate and are available in 10, 20, 40 and 80 mg atorvastatin acid equivalent strengths. LIPITOR® is indicated for prevention of cardiovascular diseases and hypercholesterolemia.

Atorvastatin calcium is a white to off-white crystalline powder that is insoluble in aqueous solutions of pH 4 and below. Atorvastatin calcium is very slightly soluble in distilled water, pH 7.4 phosphate buffer, and acetonitrile, slightly soluble in ethanol, and freely soluble in methanol. Atorvastatin calcium is susceptible to heat, moisture, light and low pH, all causing degradation from the carboxylic form to the lactone form.

U.S. Patent Application Publication No. 2003/0175338 discloses a pharmaceutical composition of atorvastatin calcium and other salts, for example atorvastatin magnesium, etc. having particle sizes less than 150 μm, such composition having improved bioavailability.

U.S. Patent Application Publication No. 2005/0032880 discloses a composition of amorphous atorvastatin calcium wherein the amorphous atorvastatin is layered around a core.

It has been surprisingly found that to match the bioavailability of a commercially available immediate release atorvastatin-containing formulation, the amount of drug released at a particular target site plays a key factor. Definite amounts of atorvastatin should be released at a specific pH in order to have a bioequivalent product. Less release of atorvastatin in the acidic pH of the stomach will result in less degradation and thereby a greater amount of atorvastatin being made available for absorption in later portion of the lower gastrointestinal tract. This subsequently would result in a composition with improved bioavailability. Hence, it poses a challenge for the formulator to formulate the dosage form in such a way that it releases a definite amount of drug at specific pH conditions of the lower gastrointestinal tract.

### SUMMARY OF THE INVENTION

The present invention relates to pharmaceutical compositions of atorvastatin or its pharmaceutically acceptable salts, solvates, hydrates, enantiomers, polymorphs or their mixtures, and processes for preparing the same, and their methods of use, treatment and administration.

In an embodiment, the invention relates to solubility enhanced forms of atorvastatin or its salts, wherein a solubility enhanced form comprises atorvastatin or its salts and at least one acid solubility enhancing excipient.

The present invention further relates to pharmaceutical compositions comprising a solubility-enhanced form of atorvastatin or its salts.

The present invention further relates to pharmaceutical compositions comprising a solubility-enhanced form of atorvastatin or its salts, such composition having a specific in vitro drug release profile in simulated acidic and alkaline pH environments of the lower gastrointestinal tract, thus ensuring desired in vivo drug release profiles.

The present invention also relates to pharmaceutical compositions of atorvastatin or its salts that use a novel formulation technique so as to achieve a product with enhanced solubility and targeted release, thereby matching the desired drug release profile in acidic as well as alkaline pH conditions of the lower gastrointestinal tract.

The invention further relates to pharmaceutical compositions comprising solubility-enhanced forms of atorvastatin or its salts, wherein solubility enhanced form of atorvastatin or its salts is in the form of a solid dispersion.

In another embodiment the invention relates to pharmaceutical compositions comprising solubility enhanced forms of atorvastatin or its salts, wherein a solubility enhanced form comprises the drug in an amorphous form.

In further embodiments the invention relates to pharmaceutical compositions comprising two discrete portions of atorvastatin or its salts.

In an embodiment the invention relates to pharmaceutical compositions comprising two discrete portions of atorvastatin or its salts wherein one portion of atorvastatin or its salts is in a solubility enhanced form.

In yet another embodiment the invention relates to process of preparing solubility enhanced forms of atorvastatin or its salts and also processes to prepare pharmaceutical compositions comprising solubility enhanced forms of atorvastatin or its salts.

In an embodiment the invention relates to methods of using pharmaceutical compositions comprising solubility enhanced forms of atorvastatin or its salts.

An embodiment of the invention provides a pharmaceutical formulation comprising two discrete portions of atorvastatin or a salt thereof.

Another embodiment of the invention provides a pharmaceutical formulation comprising a solubility enhanced form of atorvastatin or a salt thereof, comprising amorphous atorvastatin or a salt thereof in intimate mixture with at least one acid solubility enhancing excipient.

In a further embodiment, the invention provides a pharmaceutical formulation comprising two discrete portions containing atorvastatin or a salt thereof, one portion containing amorphous atorvastatin or a salt thereof in intimate mixture with at least one acid solubility enhancing excipient.

In a still further embodiment, the invention provides a pharmaceutical formulation comprising atorvastatin or a salt thereof and at least one acid solubility enhancing excipient, wherein about 10 to about 50 percent by weight of contained atorvastatin is present in an intragranular portion and the remaining contained atorvastatin is present in an extragranular portion.

In a yet further embodiment, the invention provides a pharmaceutical formulation comprising atorvastatin or a salt thereof and at least one acid solubility enhancing excipient, wherein about 15 to about 90 percent by weight of contained atorvastatin is present in intimate mixture with an acid solubility enhancing excipient.

An additional embodiment of the invention provides a pharmaceutical formulation comprising atorvastatin or a salt thereof and at least one acid solubility enhancing excipient, wherein about 10 to about 50 percent by weight of contained atorvastatin is present in an intragranular portion and the remaining contained atorvastatin is present in an extragranular portion, and about 15 to about 90 percent by weight of contained atorvastatin is present in intimate mixture with an acid solubility enhancing excipient.

### BRIEF DESCRIPTION OF THE DRAWINGS

FIG. 1 is a X-ray diffraction pattern of a solid dispersion of atorvastatin calcium and Eudragit™ E PO prepared in Example 2b.

FIG. 2 is a differential scanning calorimetry curve of a solid dispersion of atorvastatin calcium and Eudragit E PO prepared in Example 2b.

FIG. 3 is an infrared absorption spectrum of a solid dispersion of atorvastatin calcium and Eudragit E PO prepared in Example 2b.

### DETAILED DESCRIPTION

The present invention relates to pharmaceutical compositions comprising solubility-enhanced forms of atorvastatin or its salts, such composition having a specific in vitro drug release pattern in simulated acidic and alkaline pH conditions of the gastrointestinal tract, thus ensuring desired in vivo drug release profiles.

The present invention also relates to pharmaceutical compositions of atorvastatin or its salts that use a formulation technique that achieves a product with enhanced solubility and targeted release profiles.

The term “discrete portion” refers to a quantity of atorvastatin or its salts that is added in a formulation step to result in the quantity not being in simple admixture comprising another quantity of the drug.

The term “intragranular” refers to formulation components that are included before a granulation step.

The term “extragranular” refers to formulation components that are added following a granulation step.

The term “solubility enhanced form” refers to atorvastatin or its salts having a solubility that has been enhanced by any means.

The term “acid solubility enhancing excipient” refers to any excipient, which is able to dissolve in acid media and release atorvastatin from the dosage form.

Since atorvastatin calcium is susceptible to oxidation, antioxidants can be incorporated for its stabilization. Non-limiting examples of antioxidants useful in the present invention include butylated hydroxyanisole (BHA), butylated hydroxytoluene (BHT), citric acid, alpha-tocopherol, gallic acid, and the like and mixtures thereof. A mixture of atorvastatin calcium and antioxidant may be called an “atorvastatin calcium-antioxidant premix.” For example, “atorvastatin calcium-BHA premix” refers to a mixture of atorvastatin calcium and BHA in the context of the present invention.

In an aspect the invention relates to processes to prepare a premix of atorvastatin calcium and antioxidant, wherein an embodiment of the process comprises:
a) combining atorvastatin calcium with a suitable solvent and dissolving by heating to suitable temperature to form a clear solution, then cooling the solution;
b) adding an antioxidant to the above solution;
c) optionally, filtering;
d) drying the solution from step b or filtrate from step c, with or without vacuum;
e) optionally, subjecting the solid to micronization; and
f) drying the solid.

In an embodiment of the present invention, antioxidant is present in the range of less than about 5% w/w, or less than about 3% w/w, of the weight of atorvastatin calcium premix.

A further aspect of the invention relates to physicochemical properties of an atorvastatin calcium-antioxidant premix wherein the bulk density is in the range of about 0.25 to 0.45 g/ml, tapped density is in the range of 0.35 to about 0.6 g/ml, and particle size distribution is described by D₁₀ ranging from about 0.1 to 5 μm, D₅₀ from 0.1 to about 10 μm, and D₉₀ from 1 to about 20 μm.

In an embodiment the invention relates to pharmaceutical compositions comprising stabilized atorvastatin calcium-antioxidant premixes.

The present invention further relates to solubility-enhanced forms of atorvastatin or its salts, wherein a solubility-enhanced form comprises atorvastatin or one of its salts and at least one acid solubility-enhancing excipient.

In an embodiment of the present invention, a solubility-enhanced form of atorvastatin is achieved by processing atorvastatin or its salts with at least one acid solubility-enhancing excipient. Such processing comprises forming an “intimate mixture” such as a solid dispersion or solution, eutectic mixture, inclusion complex, ion-pair complex, and the like. Intimate mixtures are not obtained by merely blending solid forms of the components.

Some useful techniques for the preparation of a solubility-enhanced form of atorvastatin include, without limitation thereto, solvent evaporation, spray drying, agitated thin film drying, spray freezing, spray congealing, supercritical fluid precipitation, and other techniques known in the art. In certain embodiments, an intimate mixture is formed by removing solvent from a solution comprising atorvastatin or a salt thereof and at least one pharmaceutically acceptable polymer.

In an embodiment, the invention relates to solubility-enhanced forms of atorvastatin or its salts, in which the drug is in amorphous form.

Embodiments of the invention include solubility-enhanced forms of atorvastatin or a salt thereof, in which the drug is in amorphous form, present in one portion of a pharmaceutical formulation, with additional atorvastatin or a salt thereof present in another portion of the formulation in any of amorphous, crystalline, or mixed crystallinity forms. In certain specific embodiments, the amorphous solubility-enhanced forms of atorvastatin or a salt thereof will be present within granules that are further formulated into finished dosage forms.

“Amorphous,” for purposes of the invention, includes “substantially amorphous” drug substance having less than about 10 percent, or less than about 5 percent, by weight of a crystalline form. In certain embodiments, the drug will have less than a detectable amount of crystallinity, such as can be determined by X-ray powder diffraction analysis, and therefore is considered to be completely amorphous.

A further aspect of the invention relates to physicochemical properties of solubility-enhanced forms of atorvastatin or its salts wherein the bulk density is in the range of about 0.2 to about 0.4 g/ml, tapped density is in the range of about 0.25 to about 0.6 g/ml, and particle size distribution is described by D₁₀ ranging from about 10 to about 100 μm, D₅₀ from about 20 to about 200 μm, and D₉₀ from about 30 to about 500 μm.

In one of the embodiments of the present invention, the compositions comprise a solubility-enhanced form of atorvastatin or its salts and at least one acid solubility enhancing excipient, coated onto inert cores and then compressed into tablets, and optionally film-coated. Alternatively, atorvastatin or atorvastatin salt-coated cores, with or without a film coat, can be filled into capsules.

In one of the embodiments of the present invention, a weight ratio of atorvastatin or its salts to acid solubility-enhancing excipient ranges between about 1:0.01 and about 1:15, or between about 1:0.1 and about 1:10, or between about 1:0.1 and about 1:5, or about 1:0.5.

In another embodiment of the present invention, compositions comprise a solubility-enhanced form of atorvastatin or its salts coated onto pharmacologically inert cores, optionally mixed with other pharmaceutically acceptable excipients, and then compressed into tablets, and optionally film-coated. Alternatively, solubility-enhanced forms of atorvastatin or its salts coated onto inert cores, with or without a film coating, can be filled into capsules.

In another embodiment of the present invention, the formulation as a whole or at least partially, comprises a solubility-enhanced form of atorvastatin or its salts along with at least one pharmaceutically acceptable excipient.

In one of the embodiments of the present invention, a solubility-enhanced form of atorvastatin or its salts coated onto inert cores is mixed with a discrete portion of atorvastatin or its salts and optionally at least one pharmaceutically acceptable excipient, and then the mixture is compressed into tablets and optionally film-coated, or alternatively filled into capsules.

In another embodiment of the present invention, a weight ratio of the solubility-enhanced form of atorvastatin or its salts in a discrete portion of a composition, to atorvastatin or its salts of another discrete portion of the composition, ranges between about 1:0.1 and about 1:20, or between about 1:0.5 and about 1:15, or between about 1:1 and about 1:10, or about 1:4.

In one of the embodiments of the present invention, a solid dispersion coating comprising atorvastatin or its salts on inert cores can be achieved by techniques such as, but not limited to, brushing, rolling, dipping, spraying, layering and the like.

In another aspect of the present invention, a solid dispersion coating of atorvastatin or its salts is achieved by spraying using fluidized bed technology with Wurster, top spray or side spray techniques.

In another embodiment of the present invention, a solid dispersion coating of atorvastatin or its salts is formed on cores using top spray fluidized bed technology.

In another embodiment of the present invention, a solid dispersion coating of atorvastatin or its salts onto inert cores or excipients may be mono- or multi-layered.

In accordance with the invention, inert cores comprise pharmaceutically acceptable excipients, pellets, beads, particles or nonpareil seeds that may be water-soluble, water swellable, or water-insoluble, and organic or inorganic, or mixtures thereof.

In the context of the present invention, pharmaceutically acceptable excipients serving as inert cores comprise water-insoluble pharmaceutically inert materials, such as glass particles/beads or silicon dioxide, calcium phosphate dihydrate, dicalcium phosphate, calcium sulfate dihydrate, microcrystalline cellulose, cellulose derivatives, or soluble cores such as spheres of sugars like dextrose, lactose, anhydrous lactose, spray-dried lactose, lactose monohydrate, mannitol, starches, sorbitol, and sucrose, insoluble inert plastic materials such as spherical or nearly spherical core beads of polyvinylchloride, polystyrene or any other pharmaceutically acceptable insoluble synthetic polymeric material, and the like and mixtures thereof.

In an embodiment of the present invention, inert cores comprise a blend of lactose monohydrate and microcrystalline cellulose.

An aspect of the present invention further extends to use of an acid solubility enhancing excipient to enhance the solubility and target the release of atorvastatin.

In one of the embodiments of the present invention the acid solubility enhancing excipient comprises pharmaceutically acceptable polymers that can be water soluble, water swellable, water insoluble, pH dependent, pH independent or mixtures thereof.

Pharmaceutically acceptable polymers in the context of the invention include, but are not limited to, polyethylene glycols (molecular weight≦about 400), hydroxymethyl cellulose, hydroxyethyl cellulose, hydroxypropyl cellulose, hydroxypropyl methylcellulose, methyl cellulose, carboxymethylcellulose (CMC), sodium CMC, carboxyethyl cellulose, carboxy polymethylene, hydroxypropyl methyl phthalate, polyvinylpyrrolidone, cellulose acetate, sodium alginate, gums such as acacia gum, guar gum, tragacanth gum and xanthan gum; methacrylic acid copolymers like poly(butylmethacrylate, (2-dimethylaminoethyl) methacrylate, methylmethacrylate) Eudragit™ products designated E 100 or E 12.5 or E PO, polyvinyl acetal diethylaminoacetate (available as AEA supplied by Sankyo Co. Limited), chitosan, and the like and mixtures thereof.

Eudragit™ E is a cationic copolymer based on dimethylaminoethyl methacrylate and neutral methacrylates, having solubility in acids and used in pharmaceutical formulations to provide gastrosoluble film coatings that are soluble below about pH 5 and swellable and permeable above about pH 5. The repeating unit in the polymer has the following structure:

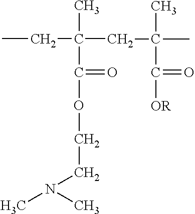

where R represents CH₃ and C₄H₉ groups and the polymer has a molecular weight about 150,000. The Eudragit E 100 product is granular, the Eudragit E 12.5 product is a 12.5% solution of E 100 in isopropanol and acetone, and the Eudragit E PO product is a fine powder made from E 100. These products are sold by Evonik Industries AG, Essen, Germany.

Organic solvents that are useful in the practice of the invention include, without limitation thereto, acetone, methanol, ethanol, isopropanol, ethyl acetate, acetone, isopropyl alcohol, methylene chloride and mixtures thereof.

In an embodiment of the invention, an organic solvent used is methanol.

Pharmaceutically acceptable excipients in the context of the present invention comprise fillers, binders, disintegrants, alkalizing agents, lubricants and glidants.

Disintegrants used in the context of the present invention include but are not limited to starches, sodium starch glycolate, sodium alginate, powdered cellulose, hydroxypropylcellulose, magnesium aluminum silicate, polacrilin potassium and mixtures thereof.

Alkalizing agents as used in the present invention increase the pH of the formulation, when such formulations are added to water. Examples of alkalizing agents include but are not limited to inorganic agents like sodium or potassium citrate, carbonate, bicarbonate, phosphate, sulfate, benzoate, ascorbate, calcium carbonate, magnesium carbonate; organic bases like buffers, meglumine, amines and mixtures thereof.

In one aspect of the present invention, the formulation may be in the form of multiparticulates, pills, pellets, granulates, capsules or tablets, which are optionally film coated.

Various parameters impacting the preparation of solid oral dosage forms include the physical parameters of an active ingredient as well as of its blends with excipients, wherein the physical parameters include flow properties, particle size (such as can be determined by sieve analyzer or Malvern particle size analyzer), bulk density and tapped density, compressibility index, Hausner ratio (determined by USP density apparatus, flow properties (determined by Flowdex apparatus), etc.

The particle size of a material is generally described in terms of D₁₀, D₅₀, D₉₀, and D_{[4,3]} used routinely to describe the particle size distribution. It is expressed as volume or weight or surface percentage. D_{[4,3]} is the volume mean diameter of the drug substance or its blend with excipients. D₉₀, for example, means that 90% by volume of the particles are below the specified particle size.

The physicochemical properties of atorvastatin calcium used in the compositions of the present invention include bulk density in the range of 0.2 to 0.35 g/ml; tapped density in the range of 0.3 to 0.6 g/ml and particle size distribution wherein D₁₀ ranges from about 0.1 to about 5 μm, D₅₀ from about 0.1 to about 10 μm, and D₉₀ from about 1 to about 20 μm.

In embodiments of the present invention, the tablets are manufactured by any process including steps such as direct compression, granulation (wet, dry or melt), melt congealing, extrusion, and any combination of two or more.

The following examples illustrate certain specific aspects and embodiments of the invention and demonstrates the practice and advantages thereof. It is to be understood that the examples are given by way of illustration only and are not intended to limit the scope of the invention in any manner.

### Example 1

Preparation of atorvastatin calcium-BHA premix.

160 g of atorvastatin calcium was added to 1600 mL of ethyl acetate followed by heating to a temperature of about 65-75° C. to obtain a clear solution, then the clear solution was cooled to a temperature of about 25 to 30° C. 0.2 g of butylated hydroxyanisole (“BHA”) was added to the above solution followed by stirring for about 5 to 10 minutes and filtration through a celite bed followed by washing the bed with 160 mL of ethyl acetate. The filtrate was passed through a agitated thin film dryer at a temperature of about 73 to 78° C. under vacuum of about 650 mm Hg. The solid material that was obtained from the agitated thin film dryer was subjected to micronization in a jet mill. The solid material was then dried using a fluid bed dryer at a temperature of 68 to 75° C. for about 4 hours. The resultant atorvastatin calcium-BHA premix is an amorphous product.

A similar process, except omitting the addition of BHA, was used to prepare neat amorphous atorvastatin calcium. This material has been used to prepare formulations in following Examples 4-6.

### Example 2

Solid dispersions of atorvastatin calcium and Eudragit E PO in different ratios.

| Ingredient | 2A | | 2B | | 2C | |
|---|---|---|---|---|---|---|
| Atorvastatin | 50 | g | 80 | g | 80 | g |
| calcium-BHA | | | | | | |
| premix (Example 1) | | | | | | |
| Eudragit E PO | 50 | g | 40 | g | 20 | g |
| Methanol # | 300 | mL | 300 | mL | 300 | mL |

# Evaporates during processing.

### Manufacturing Process:

1) Atorvastatin calcium and Eudragit E PO were dissolved in methanol using a mechanical stirrer until a clear solution was obtained.
2) The atorvastatin calcium and Eudragit E PO solution was spray dried with the following parameters:
   Inlet temperature: 48-50° C.
   Outlet temperature: 23° C.
   Pump rate: 7-12 rpm.
3) The above solid dispersions were characterized for physicochemical properties including bulk density, tapped density, particle size distribution, and saturation solubility.
   The physicochemical data are reported below in Table 1.

TABLE 1

| | Bulk | Tapped | Particle Size Distribution | | |
|---|---|---|---|---|---|
| | Density | Density | (μm) | | |
| Sample | (g/mL) | (g/mL) | D₁₀ | D₅₀ | D₉₀ |
| 2A | 0.22 | 0.42 | 50 | 113 | 237 |
| 2B | 0.22 | 0.34 | 18 | 39 | 73 |
| 2C | 0.21 | 0.34 | 13 | 30 | 55 |
| 2D* | 0.35 | 0.47 | 1 | 2 | 6 |

*Atorvastatin calcium BHA premix only.

Saturated solubility testing at 37° C. was performed by a shaken flask method wherein atorvastatin was placed into a conical flask and the flask was shaken on a rotary shaker until the medium shows resistance toward dissolving further drug substance. The solubility in the samples was determined by high performance liquid chromatograph (HPLC) after the first hour and also after 24 hours. The data are reported in Table 2, where solubility values are in mg/mL.

TABLE 2

| Medium | |
|---|---|
| | pH 6.8 |
| 0.1N HCl | Phosphate Buffer |

| Sample | 1^{st} Hour | 24 Hours | 1^{st} Hour | 24 Hours |
|---|---|---|---|---|
| 2A | 2.54 | 1.07 | 0.11 | 0.11 |
| 2B | 2.65 | 1.03 | 0.23 | 0.27 |
| 2C | 0.69 | 0.2 | 0.24 | 0.26 |
| 2D | 0.03 | 0.04 | 0.47 | 0.55 |

From the above saturated solubility data, it is observed that, compared to the atorvastatin calcium-BHA premix (2D), the presence of Eudragit increases the drug solubility in acidic media (0.1 N HCl). Increasing the solution concentration of Eudragit EP 0 from 6.67 g/L to 16.67 g/L increased the solubility of the drug from 0.2 mg/ml to 1.07 mg/mL in 0.1 N HCl.

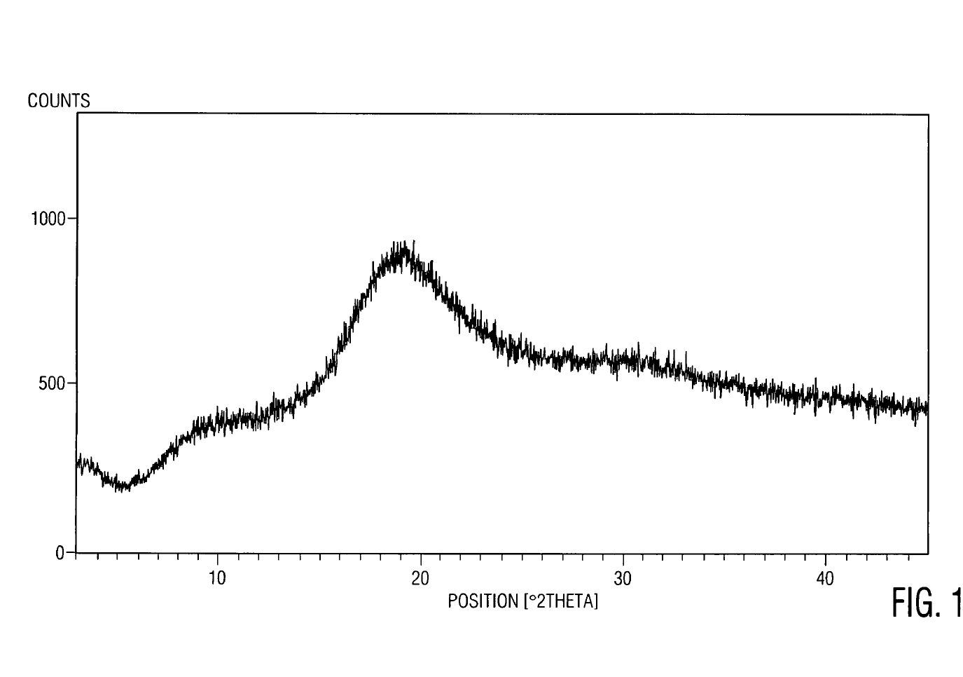

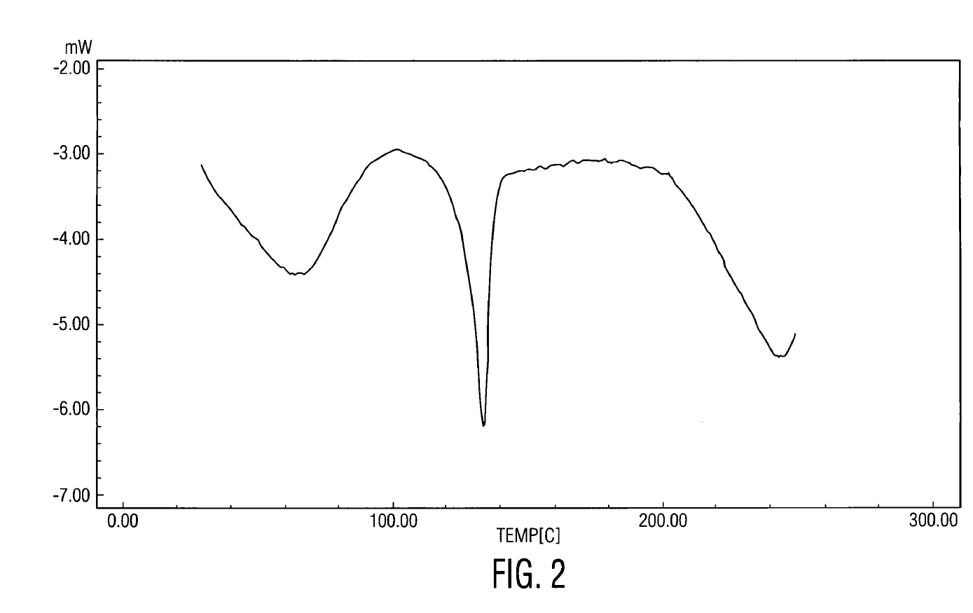

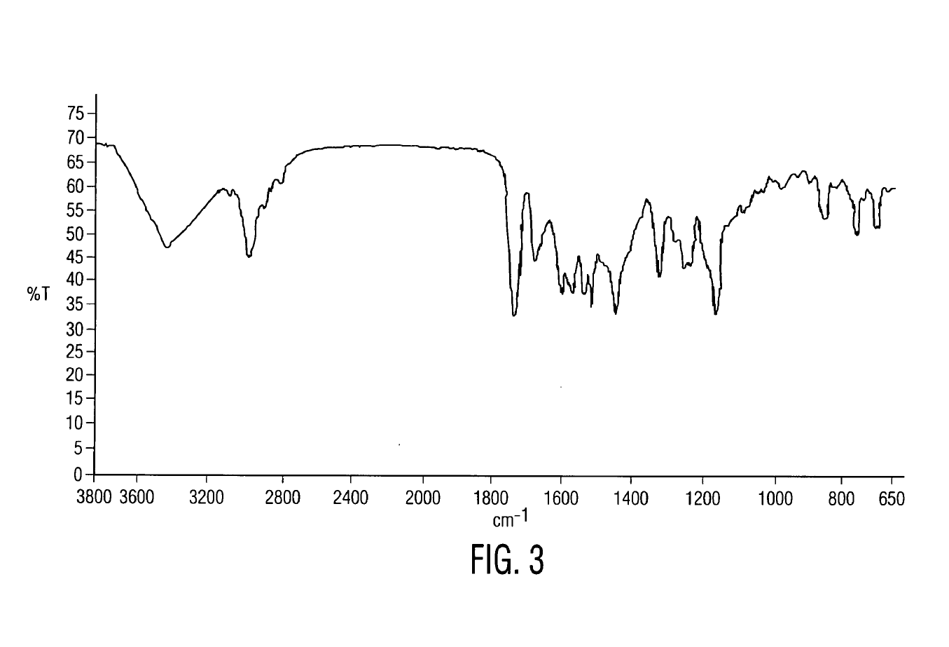

The solid dispersions, in which atorvastatin calcium was amorphous, have been further characterized by XRD (X-ray powder diffraction, using copper K-alpha 1 radiation), DSC (differential scanning calorimetry), and FTIR (infrared absorption). FIGS. 1, 2, and 3 respectively show the XRD pattern, DSC curve and FTIR spectrum for the solid dispersion of the composition 2B.

### Example 3

Atorvastatin calcium 80 mg tablets with atorvastatin calcium in intragranular material and extragranular material in the weight ratio of 30:70.

| | |
|---|---|
| Ingredient | Quantity |

GRANULATION

| | | |
|---|---|---|
| Atorvastatin calcium-BHA premix (Example 1) | 13.4 | g |
| Eudragit E PO | 6.7 | g |
| Methanol # | 125 | mL |
| Microcrystalline cellulose (Avicel PH 112) | 247.2 | g |
| Lactose monohydrate | 245.5 | g |

EXTRAGRANULAR

| | | |
|---|---|---|
| Atorvastatin calcium-BHA premix (Example 1) | 31.2 | g |
| Sodium starch glycolate | 30 | g |
| Sodium bicarbonate | 12 | g |
| Hydroxypropyl cellulose | 8 | g |

LUBRICATION

| | | |
|---|---|---|
| Magnesium stearate | 6 | g |

FILM COATING

| | | |
|---|---|---|
| Opadry White OY-58900* | 60 | g |
| Water # | 800 | mL |

# Evaporates during processing.
*Opadry White is a coating premix product manufactured by Colorcon Inc. and containing hypromellose 5 cps, titanium dioxide, and macrogol/polyethylene glycol 400.

### Manufacturing Process:

A. Drug Coating:
1. Atorvastatin calcium-BHA premix and Eudragit E PO were dissolved in methanol and the solution was stirred until it became clear.
2. Microcrystalline cellulose and lactose were sifted through a ASTM 40 mesh sieve.
3. The blend of step 2 was loaded into a fluid bed processor (FBP) bowl and solution of step 1 was sprayed using the top spray configuration of the FBP with a product temperature about 30° C.
4. The blend in the FBP was dried until the loss on drying (LOD) was less than 2% w/w, as determined at 105° C. The atorvastatin calcium is amorphous in this blend.
5. The dried granules of step 4 were sifted through ASTM 30 mesh sieve.
6. The extragranular excipients were sifted through ASTM 40 mesh sieve and mixed with the granules of step 5.

B. Lubrication and Compression:
7. Magnesium stearate was sifted through a ASTM 60 mesh sieve and was mixed with the blend of step 6.
8. The blend of step 7 was compressed to an average tablet weight 1200 mg using a 21×10 mm capsule shaped punch set.

C. Film Coating:
9. Opadry White was dispersed in water to get a uniform dispersion.
10. The tablets of step 8 were coated using an aqueous dispersion of Opadry of step 9 until a 2.5% w/w weight built-up was obtained.

Alternatively, the blend of step 8 may be filled into empty hard gelatin capsules or compressed as minitablets. The minitablets may optionally be coated as in step 10 and then filled into empty hard gelatin capsules.

In vitro release profile of the product of Example 3, in comparison with the marketed product, was obtained with the following parameters:

Media: 0.1 N HCl, 0.01 N HCl, phosphate buffer pH 5, phosphate buffer pH 6.8.

Volume: 900 mL.

Apparatus: USP apparatus 2 (Paddle) from Test 711, “Dissolution” in United States Pharmacopeia 24, United States Pharmacopeial Convention, Inc., Rockville, Md. (1999).

Speed: 50 rpm.

The data are reported in Table 3.

TABLE 3

| | Cumulative % Drug Dissolved | | | | | | | |
|---|---|---|---|---|---|---|---|---|
| | | | | | Phosphate | | Phosphate | |
| | 0.1 N HCl | | 0.01 N HCl | | Buffer pH 5 | | Buffer pH 6.8 | |
| | Lipitor ® | | Lipitor ® | | Lipitor ® | | Lipitor ® | |
| Time | 80 mg | | 80 mg | | 80 mg | | 80 mg | |
| (minutes) | Tablet | Ex. 3 | Tablet | Ex. 3 | Tablet | Ex. 3 | Tablet | Ex. 3 |
| 10 | — | — | — | — | 81 | 80 | 81 | 73 |
| 20 | 35 | 41 | 40 | 40 | — | — | 88 | 82 |
| 30 | — | — | — | — | — | — | 90 | 84 |
| 40 | 43 | 50 | 49 | 47 | 92 | 91 | — | — |
| 60 | 48 | 52 | 53 | 49 | 93 | 93 | — | — |

### Examples 4-6

Atorvastatin calcium 80 mg tablets with varying ratios of atorvastatin to polymer during granulation.

| | mg/Tablet | | |
|---|---|---|---|
| Ingredient | Example 4 | Example 5 | Example 6 |

GRANULATION

| | | | |
|---|---|---|---|
| Atorvastatin calcium (Example 1) | 84.6* | 84.6* | 84.6* |
| Eudragit E PO | 84.6 | 42.3 | 21.2 |
| Microcrystalline cellulose | 377.2 | 377 | 377.2 |
| (Avicel PH 112) | | | |
| Lactose monohydrate | 371 | 350 | 371 |
| Methanol # | q.s. | q.s. | q.s. |

EXTRAGRANULAR

| | | | |
|---|---|---|---|
| Microcrystalline cellulose | 186.5 | 250 | 250 |
| (Avicel PH 112) | | | |
| Sodium bicarbonate | 24 | 24 | 24 |
| Sodium starch glycolate | 60 | 60 | 60 |

LUBRICATION

| | | | |
|---|---|---|---|
| Magnesium stearate | 12 | 12 | 12 |

FILM COATING

| | | | |
|---|---|---|---|
| Opadry White OY-58900 | 30 | 30 | 30 |
| Water # | q.s. | q.s. | q.s. |
| Tablet weight (mg) | 1230 | 1230 | 1230 |

# Evaporates during processing.
*Quantity of atorvastatin calcium adjusted for potency.

Manufacturing process was similar to that described in Example 3.
In vitro release profile testing parameters:

Media: 0.1 N HCl.

Volume: 900 mL.

Apparatus: USP apparatus 2 (Paddle) from Test 711, “Dissolution” in United States Pharmacopoeia 24, United States Pharmacopoeial Convention, Inc., Rockville, Md. (1999).

Speed: 50 rpm.

| | Cumulative % Drug Dissolved | | | |
|---|---|---|---|---|
| Time | | | | Lipitor ® 80 mg |
| (minutes) | Example 4 | Example 5 | Example 6 | Tablet |
| 0 | 0 | 0 | 0 | 0 |
| 20 | 64 | 52 | 24 | 35 |
| 40 | 60 | 53 | 33 | 43 |
| 60 | 54 | 49 | 35 | 48 |

These results demonstrate the effect of the acid solubility enhancing excipient on atorvastatin calcium dissolution.

### Examples 7-8

Atorvastatin calcium 80 mg tablets with atorvastatin calcium in intragranular material and atorvastatin calcium in extragranular material in the weight ratios of 25:75 (Example 7) and 20:80 (Example 8).

Quantity

| | | |
|---|---|---|
| Ingredient | Example 7 | Example 8 |

GRANULATION

| | | | | |
|---|---|---|---|---|
| Atorvastatin calcium-BHA premix | 41.44 | g | 3.31 | g |
| (Example 1) | | | | |
| Eudragit E PO | 20.72 | g | 1.66 | g |
| Methanol # | 500 | mL | 32 | mL |
| Microcrystalline cellulose (Avicel | 818 | g | 81.8 | g |
| PH 112) | | | | |
| Lactose monohydrate | 417.92 | g | 84.83 | g |
| (compressible grade) | | | | |

EXTRAGRANULAR

| | | | | |
|---|---|---|---|---|
| Atorvastatin calcium-BHA premix | 124.3 | g | 13.26 | g |
| (Example 1) | | | | |
| Lactose monohydrate | 335.7 | g | 32.74 | g |
| (compressible grade) | | | | |
| Sodium starch glycolate (Type A) | 120 | g | 12 | g |
| Sodium bicarbonate | 48 | g | 4.8 | g |
| Hydroxypropyl cellulose (Klucel- | 32 | g | 3.2 | g |
| LF) | | | | |

LUBRICATION

| | | | | |
|---|---|---|---|---|
| Magnesium stearate | 24 | g | 2.4 | g |

FILM COATING

| | | | | |
|---|---|---|---|---|
| Opadry White OY-58900 | 60 | g | 6 | g |

| | | | |
|---|---|---|---|
| Water # | 450 | mL | — |

| | | | |
|---|---|---|---|
| Isopropyl alcohol # | — | 43 | mL |
| Methylene chloride # | — | 100 | mL |

# Evaporates during processing.

Manufacturing process: same as that of example 3.

The tablets prepared as above were subjected to dissolution in 900 mL of 0.1 N HCl at 50 rpm in USP apparatus II and the data are reported in Table 4.

TABLE 4

| Time | Cumulative % Drug Dissolved | | |
|---|---|---|---|
| (minutes) | Example 7 | Example 8 | Lipitor ™ 80 mg |
| 20 | 39 | 41 | 39 |
| 40 | 41 | 50 | 46 |
| 60 | 47 | 52 | 50 |

The tablets were also tested for dissolution in 900 mL of pH 6.8 phosphate buffer at 75 rpm in USP apparatus II and the data are reported in Table 4a.

TABLE 4a

| Time | Cumulative % Drug Dissolved | | |
|---|---|---|---|
| (minutes) | Example 7 | Example 8 | Lipitor ™ 80 mg |
| 5 | 63 | 76 | 85 |
| 10 | 79 | 81 | 95 |
| 15 | 84 | 82 | 98 |
| 30 | 86 | 83 | 99 |
| 45 | 89 | 83 | 101 |

### Example 9

Comparative pharmacokinetic parameters for Examples 3, 7, and 8.

Tablets were evaluated in a randomized, two-treatment, two-period, and two-sequence, single-dose crossover study involving administration to 18 healthy human volunteers under fasting conditions to determine plasma concentrations of atorvastatin. Plasma samples were withdrawn at 0.25, 0.5, 1, 1.25, 1.5, 1.75, 2, 2.5, 3, 3.5, 4, 6, 8, 10, 12, 24, 48, and 72 hours.

Wash out periods between treatments during the study were 10 days.

The following parameters were calculated:

AUC₀₋ₜ=the area under plasma concentration versus time curve, from time zero to the last measurable concentration.

AUC_{0-∞}=area under the plasma concentration versus time curve, from time zero to infinity.

Cₘₐₓ=maximum plasma concentration.

The least square mean ratios (×100) of results from Example 3, Example 7 and Example 8 formulations to a reference product (LIPITOR® 80 mg tablets, Pfizer Inc, USA) for pharmacokinetic parameter Cₘₐₓ(ng/mL), AUC₀₋ₜ (ng·h/mL), and AUC_{0-∞} (ng h/mL) values calculated from the study are reported in Table 5.

TABLE 5

| Parameter | Example 3 | Example 7 | Example 8 |
|---|---|---|---|
| Cₘₐₓ (ng/mL) | 130 | 114 | 106 |
| AUC₀₋ₜ (ng · h/mL) | 106 | 110 | 104 |
| AUC_{0- ∞} (ng · h/mL) | 106 | 110 | 104 |

1. A pharmaceutical formulation comprising two discrete portions of atorvastatin or a salt thereof.

2. The pharmaceutical formulation of claim 1, wherein atorvastatin or a salt thereof in at least one portion is in amorphous form.

3. The pharmaceutical formulation of claim 1, wherein atorvastatin or a salt thereof in a portion is present in the form of an intimate mixture with at least one acid solubility enhancing excipient.

4. The pharmaceutical formulation of claim 3, wherein an intimate mixture is formed by removing solvent from a solution comprising atorvastatin or a salt thereof and at least one acid solubility enhancing excipient.

5. The pharmaceutical formulation of claim 3, wherein an acid solubility enhancing excipient comprises a pharmaceutically acceptable polymer.

6. The pharmaceutical formulation of claim 1, wherein one portion of atorvastatin or a salt thereof is intragranular and another portion is extragranular.

7. The pharmaceutical formulation of claim 1, wherein an intragranular portion of atorvastatin or a salt thereof is amorphous.

8. The pharmaceutical formulation of claim 1, wherein a weight ratio of two discrete portions ranges from about 1:0.1 to about 1:20.

9. The pharmaceutical formulation of claim 1, wherein a weight ratio of two discrete portions ranges from about 1:0.5 to about 1:15.

10. The pharmaceutical formulation of claim 1, wherein a weight ratio of two discrete portions ranges from about 1:1 to about 1:10.

11. The pharmaceutical formulation of claim 1, wherein a weight ratio of two discrete portions is about 1:4.

12. A pharmaceutical formulation, comprising amorphous atorvastatin or a salt thereof in intimate mixture with at least one acid solubility enhancing excipient.

13. The pharmaceutical formulation of claim 12, wherein a weight ratio of atorvastatin or a salt thereof to acid solubility enhancing excipient ranges from about 1:0.01 to about 1:15.

14. The pharmaceutical formulation of claim 12, wherein a weight ratio of atorvastatin or a salt thereof to acid solubility enhancing excipient ranges from about 1:0.1 to about 1:10.

15. The pharmaceutical formulation of claim 12, wherein a weight ratio of atorvastatin or a salt thereof to acid solubility enhancing excipient ranges from about 1:0.1 to about 1:5.

16. The pharmaceutical formulation of claim 12, wherein a weight ratio of atorvastatin or a salt thereof to acid solubility enhancing excipient is about 1:0.5.

17. The pharmaceutical formulation of claim 12, wherein an acid solubility enhancing excipient comprises a methacrylic acid copolymer or a derivative thereof.

18. A pharmaceutical formulation comprising two discrete portions containing atorvastatin or a salt thereof, one portion containing amorphous atorvastatin or a salt thereof in intimate mixture with at least one acid solubility enhancing excipient.

19. The pharmaceutical formulation of claim 18, wherein a portion containing amorphous atorvastatin or a salt thereof in intimate mixture with at least one acid solubility enhancing excipient is intragranular.

20. A pharmaceutical formulation comprising atorvastatin or a salt thereof and at least one acid solubility enhancing excipient, wherein about 10 to about 50 percent by weight of contained atorvastatin is present in an intragranular portion and the remaining contained atorvastatin is present in an extragranular portion.

21. A pharmaceutical formulation comprising atorvastatin or a salt thereof and at least one acid solubility enhancing excipient, wherein about 15 to about 90 percent by weight of contained atorvastatin is present in intimate mixture with an acid solubility enhancing excipient.

22. A pharmaceutical formulation comprising atorvastatin or a salt thereof and at least one acid solubility enhancing excipient, wherein about 10 to about 50 percent by weight of contained atorvastatin is present in an intragranular portion and the remaining contained atorvastatin is present in an extragranular portion, and about 15 to about 90 percent by weight of contained atorvastatin is present in intimate mixture with an acid solubility enhancing excipient.

23. The pharmaceutical formulation of claim 21, wherein atorvastatin or a salt thereof in intimate mixture with an acid solubility enhancing excipient is amorphous.

24. The pharmaceutical formulation of claim 22, wherein atorvastatin or a salt thereof in intimate mixture with an acid solubility enhancing excipient is amorphous.
